(19) United States
(12) Patent Application Publication
(10) Pub. No.: US 2007/0060876 A1
(43) Pub. Date: Mar. 15, 2007

(54) DUAL CHAMBER CONTAINER WITHOUT BY-PASS

(76) Inventors: Stefan Bassarab, Biberach (DE); Alexander Bauer, Biberach (DE); Nicole Denkinger, Biberach (DE); Patrick Garidel, Norderstedt (DE); Markus Hemminger, Biberach (DE); Hans-Joachim Kern, Mittelbiberach (DE); Andreas Langer, Maselheim (DE); Ingo Presser, Biberach (DE)

Correspondence Address: MICHAEL P. MORRIS;BOEHRINGER INGELHEIM CORPORATION, 900 RIDGEBURY ROAD, P. O. BOX 368, RIDGEFIELD, CT, 06877-0368, US

(21) Appl. No.: 11/464,492

(22) Filed: Aug. 14, 2006

(30) Foreign Application Priority Data
Aug. 13, 2005 (DE) 10 2005 038 497

(51) Int. Cl. A61M 37/00

(52) U.S. Cl. 604/88

(57) ABSTRACT

The invention relates to a double chamber container for holding and combining two separate components, at least one of which is liquid, comprising a cylindrical body (15) with a closure (20, 60) at each of the two ends (5, 70) of the body (15), an upper closure (60) at the liquid component end and a lower closure (20) at the solid component end, and a separating stopper (40) in the cylindrical body (15) as a seal between the two chambers (30, 50), the separating stopper (40) having a height H and the upper closure (60) at the liquid component end being movable by the application of pressure, and comprising on its underside a hollow needle (45) with a length L having at least one opening (42) which is provided at the end opposite the tip, where H<L.

This application claims benefit under 119(a) of DE 10 2005 038 497, which was filed on Aug. 13, 2005, and which is incorporated herein by reference in its entirety.

### BACKGROUND OF THE INVENTION

1. Field of the Invention

The present invention relates to a double chamber container without a bypass, a method of filling it and the use thereof.

2. Description of the Prior Art

There are pharmaceutical compositions which in their ready-to-use form very rapidly lose their efficacy. To enable these compositions to be used in spite of their short shelf life, two-compartment systems have been developed, for mixing them immediately before use.

Thus, pharmaceutical preparations which cannot be used in solution over long periods may be made durable by lyophilization, for example, and possibly stored away from light. The dry substance is only dissolved again, i.e. reconstituted, immediately before use.

As is known, lyophilizates are also used in ready-prepared syringes with two-chamber systems—the lyophilizate and solvent being stored separately and only combined just before use. Two-chamber prepared syringes of this kind have already been described in “Die Lyophilisierung von Arzneimitteln in Fertigspritzen”, H. Vetter, Die Pharmazeutische Industrie, 46th year, 1984, No. 10, p. 1045-1049. These ready-prepared syringes were developed in order to do away with the usual major effort of handling two sterile sealed containers and a syringe and to rule out the risk of confusion, for example, with unlabelled disposable syringes.

The ready-prepared syringes are usually cleaned, siliconized, and provided with a guard for the cannula. The siliconization for coating the primary packaging means is carried out so that the rubber stopper can easily be pushed into the glass cylinder. Both the rubber stopper and the inside of the glass cylinder are covered with a silicon oil. Then the ready-prepared syringes can be pre-sterilized in batches and filled under sterile conditions. They are then fitted with a cylinder stopper and finished, at which time the plunger rods are fitted. Then they are labeled and packaged. The filling and sealing are carried out from the rear end of the syringe, as the opening at the cannula end is too small.

Specifically, ready-prepared syringes of this kind are constructed so that each of the components is housed in its own chamber, the chambers being arranged axially behind one another and only brought into contact with one another immediately before use, so that the liquid component in one chamber can flow over to the dry component in the other chamber. This produces the desired mixture. It is known to provide a bypass for this purpose, for example in the form of a widening in the internal diameter of the glass cylinder in the syringe, through which the liquid component can flow when a stopper separating the two chambers is pushed so far into the bypass that the liquid component can flow around the stopper.

In the prior art attempts were made to find improved solutions enabling the two-component systems to be mixed without touching any part of the device or the components during preparation. There are numerous proposals along these lines, while in the present case only those systems which do not have a bypass as described above are of interest.

The patents described in the following paragraphs, namely DE 32 13 072, DE 1 809 892, U.S. Pat. No. 5,785,683, U.S. Pat. No. 6,602,223, EP 1 038 543, DE 25 46 495, and U.S. Pat. No. 3,810,469, are incorporated herein by reference in their entireties.

For example, DE 32 13 072 describes a syringe which is subdivided into two compartments for holding a powdered medicament and its solvent, the mixing being carried out by withdrawing the plunger rod of the syringe, so that the solvent can flow around the plunger.

In DE 1 809 892 a two-chamber syringe is described, the filling of which is made easier by the fact that the liquid is added through a filling channel provided in the plunger rod.

In U.S. Pat. Nos. 5,785,683 and 6,602,223 the process of mixing the two components in the syringe is achieved using specially designed valves which can be controlled by moving the plunger.

Moreover, EP 1 038 543 discloses a twin-chamber syringe for medical purposes which contains a plunger rod with projecting latching elements which are arranged in diametric pairs and/or at a rotation angle of 90° to ensure a controlled mixing process and sequential administration of the mixture.

It is also known from DE 25 46 495 to connect a syringe via a special stopper to a container which is sealed off by a second stopper, which is expelled before the mixing process.

All the systems described have the disadvantage of being very complex in construction, based on specially designed valves or plunger rods. DE 25 46 495 in particular has, in addition to a complex structure which connects a syringe and a container via a connecting member, the serious disadvantage that 2 containers have to be joined together, the actual weak point being the connecting member, which may slip or fall off during lengthy storage. Therefore there is the risk of contamination from outside. Moreover, this design is very bulky and therefore not very easy to store and handle.

In addition U.S. Pat. No. 3,810,469 describes a multi chamber ampoule having a needle, in which the chambers are separated by two membranes. Using one and the same ampoule the fluid contained therein can be freeze-dried, a liquid and a solid substance can be mixed together, and the mixture can then be injected subcutaneously. Accordingly, an exceptionally complicated structure is present, in which the chambers are sealed by partly drilling through one of the membranes with the needle, which can be fixed therein. When the solid and liquid substances are mixed together a second membrane is pierced by the needle and the liquid is forced up into the upper chamber from the lower one. A particular disadvantage is that the needle for sealing off the chambers has to be very accurately adjusted and secured as otherwise it will damage the membrane and premature mixing may take place. This makes handling particularly difficult. The high costs of producing such a complicated ampoule mean that it has hardly been used at all on an industrial scale.

There is therefore still a need for a device which is easy to handle, for the reliable storage and mixing of two-component systems, at least one of which is in liquid form. At the same time, contamination from outside should be prevented as far as possible, i.e. the mixing should take place without removing the components from the sterile interior of the device and without compromising the sterility. The device should also be easy to store. In addition, an easy method of filling such a device should be provided. The device and the method should also be suitable for implementation on an industrial scale.

### SUMMARY OF THE INVENTION

The objective described above is achieved by the features of claim 1. According to this, a double chamber container for holding and combining two separate components, at least one of which is liquid, is provided, comprising
a cylindrical body with a closure at each of the two ends of the body, an upper closure at the liquid component end and a lower closure at the solid component end, and
a separating stopper in the cylindrical body as a seal between the two chambers, the separating stopper having a height H and the upper closure being movable at the liquid component end by the application of pressure, and comprising on its underside a hollow needle with a length L having at least one opening which is provided on the end opposite the point, where H<L.

### DESCRIPTION OF THE FIGURES

The accompanying Figures illustrate the device according to the invention and the procedural teaching which is to be carried out according to the invention without restricting the invention thereto. Specifically:

FIG. 1 is a schematic representation of a container according to the invention with an open end before lyophilization;

FIG. 2 is a schematic representation of a container according to the invention in the filled, sealed state;

FIG. 3 is a schematic representation of a container according to the invention during the mixing of the two components and

FIG. 4 shows schematic representations of possible stopper shapes.

### LIST OF REFERENCE NUMERALS

5 end on the solid side (end B)

10 container

15 cylindrical body

20 closure, mouth

30 chamber containing solid component

33 lyophilization solution

40 separating stopper of height H

42 opening

45 hollow needle of length L

50 chamber containing liquid component

60 closure, stopper

70 end on the liquid side (end A)

### DETAILED DESCRIPTION OF THE INVENTION

The objective described above is achieved by the features of claim 1. According to this, a double chamber container for holding and combining two separate components, at least one of which is liquid, is provided, comprising
a cylindrical body with a closure at each of the two ends of the body, an upper closure at the liquid component end and a lower closure at the solid component end, and
a separating stopper in the cylindrical body as a seal between the two chambers, the separating stopper having a height H and the upper closure being movable at the liquid component end by the application of pressure, and comprising on its underside a hollow needle with a length L having at least one opening which is provided on the end opposite the point, where H<L.

The present invention therefore describes a single-chamber container in which a double chamber system without a bypass is provided. The liquid component changes the chamber through an opening in an internally mounted hollow needle which penetrates the separating stopper. Accordingly, an internal “bypass” for mixing the two components, which is integrated in the separating stopper between the two chambers, is only formed immediately before the mixing. As a result, a bypass which is conventionally formed in ready-made double-chamber syringes or carpules is mounted in practically integrated manner.

When the closure containing a hollow needle, designed according to the invention, is used, the closure is pushed towards the separating stopper by the application of an external force, particularly a force to be exerted manually, and at the same time pressure is exerted on the liquid end, causing the hollow needle to be pressed into or through the separating stopper, until the liquid component can access the chamber containing the solid component. The two components can thus be mixed together without compromising the sterile conditions of the double chamber container.

Thus, according to the invention one chamber may contain a liquid and the other a solid. The invention is not restricted as to the nature of the two components, provided that at least one component is liquid. For example, a solid such as a solid powdered pharmaceutical composition, preferably a lyophilizate, i.e. a freeze-dried medicament, may be present. In lyophilization or freeze-drying, a liquid product is frozen and then the frozen product is dried by sublimation. Preferably, the liquid component may be for example a dissolving or dispersing medium for the solid; it may be for example a reconstituting medium for the lyophilizate. If the solid is a pharmaceutical composition, an injectable solution may be prepared, for example, in either dissolved or dispersed form. The solvent is preferably water, but may also be some other solvent or a mixture of solvents.

The cylindrical body according to the invention is an essentially elongate hollow body with two open ends which has preferably been formed integrally, i.e. made in one piece, and subdivided by the separating stopper into chambers. The “cylindrical” body need not necessarily be cylindrical in shape, although this is the most common shape. Any other geometric shape for an elongate hollow body is possible, such as angular or oval, for example, in which case the closures and separating stopper and the like must be matched to the chosen shape. The material of which the cylindrical body consists or which it contains is not particularly restricted according to the invention. The container may be selected from plastics or glass. Glass is preferred on account of its transparency and its compatibility with numerous medical formulations. The cylindrical body therefore preferably consists or glass or contains glass, as this produces the least effect on the components contained therein and the body is preferably transparent. For particular requirements, however, other materials may be suitable, such as special plastics or the like. Medical safety is particularly important, as it is desirable that there be as little interaction as possible with the medium contained therein.

Preferably, the cylindrical body has the same diameter over its entire length.

The double chamber container further comprises 2 closures, one of which is provided at the solid (lower) end and the other at the liquid (upper) end. The closure means are not further restricted, provided that the closure device at the liquid end closure device is movable.

The closures may be selected for example from stoppers, membranes, sealing discs, optionally with covering caps, flanged caps or the like. Preferably a stopper with a hollow needle is used as the upper closure. However, any other closure known in the art may be used, provided that it can be moved by the application of pressure and comprises a hollow needle. It is particularly convenient if the upper closure at the liquid end of the container is a stopper which may be made of plastics or similar materials. Examples of materials include rubber or rubber-like elastic material such as elastomers, thermoplastics, and elastomeric thermoplastics.

The lower closure at the solid end may preferably be a pierceable membrane with an outer cap or a disc for sealing it, which is flexible, for example, and may comprise a flanged cap, for example. A removable cover may also be provided. If the container is a carpule or ampoule, a rubber closure may be provided at one or both ends, which can be pierced with an injection needle. However, any other closure known to the skilled man may also be used. It goes without saying that the closures may also be removably fixed. In any case, care must be taken to ensure that the closures are sealed and sterile.

The hollow needle is not particularly restricted within the scope of the invention. It may be a conventional hollow needle or cannula used in the medical field. It is a hollow tube that may have a point at one end, is optionally ground at an angle, and optionally has a sharpened tip to enable it to penetrate better.

The hollow needle is preferably arranged in the centre of the base of the upper closure or stopper. However, this is not essential. The hollow needle may be provided virtually anywhere in the base, but a central position is preferred. If a stopper is provided, e.g. made of a soft elastic material, the hollow needle may be integrated in the base thereof at the desired location. The force-transmitting area should be as large as possible in order to prevent the penetration of the end stopper.

The material of the hollow needle is usually metal or plastics. The internal diameter of the hollow needle may be suitably selected in accordance with the liquid component and in this way the speed of passage may be controlled. Preferably the needle has an inner diameter of about 0.2 mm (29 G) to about 1.6 mm (14 G), but smaller or larger diameters are also possible.

The opening in the hollow needle is provided at the end opposite the tip, i.e. as far upwards and possible in the hollow needle at the outermost upper end or at some other suitable location, and is not restricted to a particular shape or size. For example, a round, oval, triangular, or rectangular opening may be used. This may be selected in accordance with the intended use. Preferably the opening has a diameter of about 0.2 mm to about 1.4 mm, but smaller or larger diameters are also possible.

It is particularly preferred to adjust the size of the opening and the inner diameter of the hollow needle such that the desired overflowing of the liquid component takes place readily and at the desired speed.

The hollow needle may have not only one but also two, three or more openings.

It is particularly preferred according to the invention if the hollow needle for the mixing process is pressed so far into the separating stopper that the opening is still at least partly located in the chamber containing the liquid component. This makes it easier for the liquid component to flow through.

The hollow needle is preferably mounted in the centre of the upper closure or stopper. However, this is not necessary in every case. According to the invention it is possible to use not only one hollow needle but a plurality of hollow needles.

The separating stopper arranged in the cylindrical body defines the size/volume/dimensions of the two chambers and functions as a liquid barrier device for the liquid component, which is prevented from passing into the other chamber. The shape of the separating stopper is not particularly restricted. It has a suitable three-dimensional shape so that the two chambers are sealed off from one another. Admittedly, the separating stopper is primarily arranged in stationary manner in the cylindrical body and therefore cannot be moved when force is applied to the chamber containing the liquid component, but it is also possible to press it down as far as the solid component by the suitable application of pressure.

The separating stopper is preferably made of an elastic and flexible material such as rubber, caoutchouc, natural and synthetic rubber, plastics, such as elastomers, thermoplastics, thermoplastic elastomers and the like. The material of the separating stopper should provide a guaranteed seal between the two chambers but should at the same time be easy for a hollow needle to penetrate.

According to a preferred embodiment according to the invention the separating stopper is of a suitable shape, size and/or material that on the one hand will prevent it from being pushed out of its fixed and defined position in the cylindrical body but on the other hand is soft and flexible enough to enable the hollow needle to pass through. Conveniently the shifting of the separating stopper may be achieved by the corresponding provision of a suitable shape with (adhesive) bumps, lips, beads, or webs and/or the choice of a suitable diameter, if desired.

In the present invention the term “form” is intended to refer to the outer shape or geometry. The term “size” is intended to refer to the dimensions, i.e. the ratios of magnitude.

The separating stopper may be of any suitable shape; preferred shapes are cylindrical shapes, cylindrical shapes with rounded sides, dumbbell-shaped, cuboid, conical, truncated frustum, or conical shapes.

The separating stopper preferably has an outer diameter which is greater than the inner diameter of the cylindrical body, so that sufficient pressure is built up between the inner wall and shaped member to close off the interface but so that the latter is movable in the container under the effect of force, depending on the particular application.

If the liquid is put under pressure, the separating stopper continues to adhere to the inner wall of the cylindrical body by frictional forces. If the frictional adhesion of the separating stopper to the wall of the cylindrical body is not sufficient for a particular application, to prevent accidental movement, the latter may additionally be provided with projections such as small (sealing) beads, lips, bumps, or surfaces to adhere to the inner wall of the cylindrical body. The pressure therefore does not increase in the other chamber. As a result, a differential pressure is produced between the two chambers, as a result of which, once the access channel has been formed, the liquid component is able to flow through the opening from one chamber into the other and the two components are mixed.

The mixing may for example be the reconstitution of a lyophilizate. During this step, no other part of the container comes into contact with the liquid or solid component.

The height of the separating stopper is selected to be less than the length of the hollow needle L, so that the hollow needle can readily pass through the separating stopper. It may be advantageous if the opening in the hollow needle is arranged so that after total penetration it is still above the separating stopper, i.e. in the chamber containing the liquid component. The opening is therefore preferably in the part by which the length L exceeds the height H of the separating stopper, where L>H.

According to a particularly preferred embodiment of the invention, spacers and/or stops are provided on the hollow needle at the upper closure and/or at the separating stopper, to make it difficult or impossible for the opening of the hollow needle to penetrate fully into the separating stopper. These may consist for example of small bumps.

Preferably the double chamber container is not a syringe but a vessel for separately storing two substances, such as an ampoule or carpule, which is intended for single or multiple uses.

The measurements of the double chamber container depend on the volume of the solution which is to be produced; in human medicine, volumes of 10 ml are rarely exceeded, which means that volumes of up to about 20 ml are sufficient. In exceptional cases and for veterinary use however it is possible to exceed these volumes by a long way.

The invention also relates to a method of filling the double chamber container according to the invention, comprising the following steps:
(1) sealing a cylindrical body at the lower end;
(2) filling the cylindrical body with a lyophilization solution;
(3) lyophilizing the cylindrical body in the lyophilizer to obtain a lyophilizate cake;
(4) putting a separating stopper above the lyophilizate cake, the separating stopper having a height H;
(5) filling the cylindrical body with reconstitution medium above the separating stopper;
(6) sealing the cylindrical body with a closure comprising a hollow needle, wherein the closure is movable at the liquid component end by the application of pressure and comprises, on its underside, a hollow needle with a length L having at least one opening which is provided at the end opposite the tip, where H<L, and the hollow needle is directed towards the separating stopper.

The process will hereinafter be described in detail; any individual features described in relation to the process also apply accordingly to the double chamber container and vice versa.

In a first step of the filling process according to the invention the lower end or the mouth of the cylindrical body is sealed. The lower end may for example also have a taper. The closure may be for example a stopper, a membrane, a disc, particularly a sealing disc, optionally with a cover, such as a covering cap or flanged cap, or the like. The closure is not particularly restricted as long as it provides a suitable seal, is inert with respect to the medium which is to be added, and meets the conditions of sterility. The lower seal is most particularly preferably a pierceable membrane, a rubber stopper, or an elastic disc, optionally with a flanged cap. It is also possible to provide a removable closure. If the container is a carpule or ampoule, a rubber closure may be provided which can be pierced with an injection needle. However, any other seal known in the art may also be used.

Then either a solid component or a liquid containing the solid component is added. In the latter case the cylindrical body is then filled with a lyophilization solution through the open lower end of the cylindrical body (step 2). This may be any desired solution which is to be lyophilized. It may for example be a medicament which is to be made durable by the lyophilization process.

The lyophilization is carried out in step (3) in a manner known in the art, with the solvent escaping through the open lower end of the cylindrical body. The lyophilizer is of a standard commercial kind, the process parameters of which are automatically regulated, for example.

After the lyophilization the cylindrical body is sealed in step (4), which may also be carried out in the lyophilizer, by pressing a separating stopper above the lyophilizate cake into the cylindrical body. This separating stopper may preferably be placed centrally in the cylindrical body, but may also be positioned anywhere, depending on the particular application and the desired size of the two chambers. The closing of the cylindrical body is most preferably carried out under vacuum, so that the separating stopper can readily be positioned in the desired location. In this way the separating stopper can be pressed far enough into the cylindrical body without causing excess pressure in the chamber which has already been sealed.

Then the sealed container is taken to a filling station, for example, where in step (5) it is filled with reconstituting medium through the top end of the cylindrical body which is now open again, i.e. the solvent or dispersing medium is poured into the upper chamber. After the container has been filled with reconstituting medium, i.e., a dissolving or dispersing medium for the lyophilizate, the container is fitted with a closure which comprises a hollow needle directed towards the separating stopper (step (6)). For example, a stopper may be used as the upper closure. Examples of materials for the stopper are rubber or rubber-like elastic material such as elastomers, thermoplastics, elastomeric thermoplastics, etc.

It is particularly expedient if the stoppers and/or closure members are supplied and inserted by means of a washing and sterilizing device or an autoclave along sterile corridors. After the container has been sealed it is taken out of the sterile area through an airlock; finally it is labeled and packaged. It will be understood that in this process all the surfaces and equipment are designed for aseptic operation.

The detailed explanations provided above regarding the double chamber container also apply correspondingly to the process for filling it.

The invention also provides a method of mixing 2 separate components, at least one of which is liquid, in a double chamber container, comprising a cylindrical body with a closure at each of the two ends of the body, an upper closure at the liquid component end and a lower closure at the solid component end, and a separating stopper in the cylindrical body as a seal between the two chambers, the separating stopper having a height H and the upper closure at the liquid component end being movable by the application of pressure and having, on its underside, a hollow needle with a length L having at least one opening which is provided at the end opposite the tip, where H<L, comprising the steps of:
applying pressure to the movable upper closure until the hollow needle has completely penetrated the separating stopper and
allowing the liquid component to overflow through the opening and the hollow needle to the solid component, to obtain a mixture.

Preferably the upper closure is a stopper, especially a rubber stopper, which comprises a hollow needle. The two components are preferably mixed together by holding the cylindrical body vertically, i.e. with the lower closure, i.e. the solid end, at the bottom. Preferably the closure at the solid end comprises a sealing disc or a stopper optionally having a cover, such as a flanged cap, but it is also possible to use any other suitable closure.

The pressure on the upper closure or stopper may be exerted using the fingers or a suitable punch. Pressure is applied until the upper closure abuts on the separating stopper. It has been found that the gap remaining between the two stoppers is sufficient to allow the liquid component to pass to the opening until all the liquid has flowed over.

The separating stopper may be pressed down onto the solid component during the application of pressure and during the penetration through the hollow needle, but this is not always necessary. It depends for example on the choice of material, the diameter of the hollow needle, the location of the separating stopper in the cylindrical body.

According to one embodiment of the invention the hollow needle is pressed into the separating stopper in order to carry out the mixing process, to the point where the opening is still at least partly located in the chamber containing the liquid component. However, this is not always essential because, as explained previously, the movable upper closure and the separating stopper can also come into direct contact without totally preventing the passage of liquid. This is also connected with the fact that as the pressure increases with the displacement of the upper closure, the liquid component flows all the more rapidly into the adjacent chamber, as a result of the ever increasing pressure difference, so that when the upper closure and separating stopper make contact the liquid still present is forced downwards.

Preferably, this overflow may additionally be influenced and chosen by the choice of the shape of the opening and/or the size and internal diameter of the hollow needle, so that the speed of passage of the liquid component into the other chamber can be controlled.

According to another embodiment, spacers may be provided on the upper closure which carries the hollow needle and/or on the separating stopper, and/or stops may be provided on the hollow needle above the opening to prevent or at least impede total penetration of the opening into the separating stopper.

The invention further relates to the use of the container according to the invention in human and veterinary medicine.

The present invention has a number of advantages:

The inventive achievement is that a liquid component is transferred between two chambers by means of an internal hollow needle. This creates a double chamber system in a single chamber container. Thus, this internal “bypass” enables the two component system to be stored without any problems without any premature mixing and hence loss of efficacy of the components. The two-component system provided according to the invention can be stored in its sterilized, pre-filled, ready-to-use state. The two components are mixed together after storage, immediately before use. The double chamber container can be thrown away after use.

Thus, a device is provided by means of which the desired solution is produced immediately before use, resulting not only in a fast and reliable system, but also ease of manufacture and filling. This is an “all-in-one” solution. In other words, the vessel has two chambers which are separated from one another by a stopper.

Using the double chamber container according to the invention it is possible to carry out mixing, e.g. reconstitution of a lyophilizate, in a sealed two-chamber system by simple means, without having to provide the container per se with an additional external or internal bypass and/or without having to break the seal of the system in order to carry out the mixing, thereby opening it up to undesirable external microbial, chemical or physical influences.

Accordingly, there is no risk of contamination by foreign bodies which might penetrate from outside, such as bacteria, particles of dirt, splinters, etc. The container can be used as primary packaging and stored in its clearly labeled form. The use of two-component systems, particularly lyophilized preparations, can thus be made simpler.

There is also the possibility, by suitable dimensioning of the opening in the separating stopper and/or the inner diameter of the hollow needle, to adjust the flow velocity or quantity of liquid flowing through per unit of time accordingly. In this way a correspondingly slow entry or rapid overflowing from one chamber into the other can be controlled by the channel size and/or the size of the opening. There is no need to restrict the overflow speed of the liquid component using additional equipment, which would involve considerable expense. Problems that arise in syringes, when too much pressure is applied to the stopper and as a result the liquid component surges forward too quickly over the bypass to the chamber containing the dry substance and as a result any substance which has already been dissolved leaves the cannula of the syringe, can be avoided entirely. Concomitant undesirable contamination of the user and loss of the dissolved substance and hence a dosage which can no longer be precisely controlled are thus prevented.

A further advantage of the invention is that the double chamber container required for this system has no outer irregularities, in the form of a bypass with one or more protuberances and can therefore be machined more easily and reliably on the usual processing equipment. This has advantages in processing, for example, during filling, lyophilization, the fitting of the closures, labeling, and also handling, where a bypass may represent a possible frangible point on the container and requires special technologies which are not necessary here.

The above description discusses a number of variations and suggests a range of possible modifications which will be immediately apparent to the skilled man.

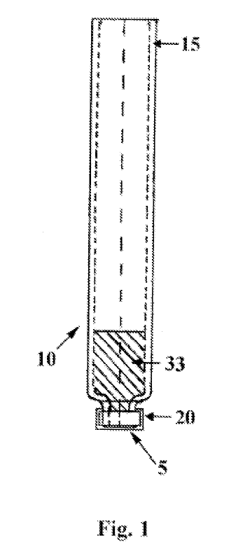

FIG. 1 is a schematic representation of a container 10 according to the invention immediately before the lyophilization is carried out. A cylindrical body 15 is shown which may be made of glass, for example. It is provided at one end 5 with a closure 20. This may be a sealing disc or a stopper with a flanged cover, for example. The cylindrical body 15 is filled with a lyophilizing solution 33 which is then lyophilized in the lyophilizer in the usual way. Above the lyophilizate cake 30 obtained a separating stopper 40 is then inserted and a reconstitution medium 50 is added. Then the cylindrical body 15 is closed off by means of a closure modified with a hollow needle, such as an end stopper 60, at the upper end 70 of the cylindrical body 15, the hollow needle 45 being directed towards the separating stopper. The hollow needle is preferably mounted in the centre of the base of the end stopper 60 and has at its upper end, or additionally in the centre (not shown), one or more openings 42 which allow liquid to pass into the hollow needle.

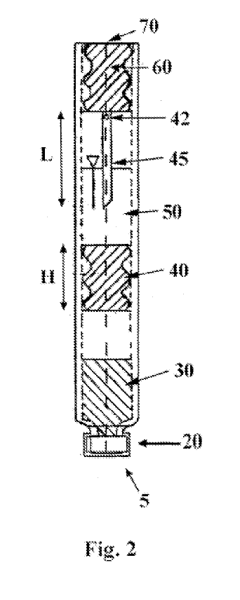

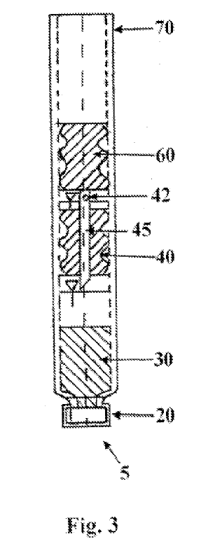

FIGS. 2 and 3 diagrammatically show the function of a double chamber container 10 according to the invention in the starting position (FIG. 2) and in the mixing position (FIG. 3). Specifically, a double chamber container 10 according to the invention is shown which is made up of a cylindrical body 15, made of glass, for example, a solid-end closure 20 at the open end 5, and a liquid-end closure 60 at the open end 70 of the cylindrical body 15. The closure 20 constitutes a rubber closure in the present case, which may be a pierceable membrane with an outer cap, which is flanged, for example, over the outer edge at the emptying end. A removable disc connected to the outer cover may be provided over this. Obviously it is also possible to provide any other closure known to the skilled man, particularly a removable closure.

The upper closure 60 may, for example, be a stopper which may be made of a suitable material, such as rubber, plastics, or the like. The nature of this closure is not limited further, provided that it enables a pressure to be applied to the liquid component in the chamber 50, and has a hollow needle, so that the separating stopper 40 can be penetrated.

The separating stopper 40 subdivides the container 10 into two chambers 30 and 50, the chamber 30 containing a solid component such as, for example, a lyophilizate, while the other chamber 50 contains a liquid component, such as a reconstitution medium for the solid component. The closure or end stopper 60 has a hollow needle 45 with an opening 42, the height H of the separating stopper being less than the length L of the hollow needle. The hollow needle 45 may for example be a cannula with a chamfered and/or pointed tip.

If pressure is applied to the closure or end stopper 60, it moves in the direction of the separating stopper 40. The hollow needle 45 meanwhile comes into contact with the separating stopper 40, which as a result of the frictional forces with the wall of the cylindrical body 15 initially remains where it is.

The separating stopper 40, which separates the chamber 50 with the liquid component (end A) of the two-chamber system from the chamber 30 with the solid component (end B), is preferably constructed like a conventional separating stopper at its side face which forms a seal with the wall of the cylindrical body 15. If the adhesive friction of the separating stopper 40 on the wall of the cylindrical body 15 is not sufficient for the intended purpose, i.e. to prevent undesirable slipping or movement of the separating stopper 40, this may preferably additionally be held by means of small bumps (not shown) on the inner surface of the cylindrical body 15.

As pressure continues to be applied the hollow needle 45 finally penetrates the separating stopper 40 and thereby opens up a passage for the liquid component through the opening 42 into the chamber 30. For example, a lyophilizate may be reconstituted.

Preferably the opening 42 in the hollow needle 45 is still fully within the chamber 30 after the penetration of the separating stopper 40. However, the opening 42 may also have penetrated partly or totally into the separating stopper 45.

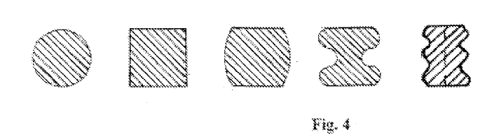

The stopper 60 and separating stopper 40 may be of any suitable size and shape. For example, as shown in FIGS. 2 and 3, they may have various convexities in the form of large bumps. However, they may also be formed in a cylinder or a cylinder with a spherical outer surface or in a dumbbell shape with two sealing surfaces, as shown by way of example in FIG. 4.

The foregoing description of the Figures serves to illustrate the apparatus according to the invention and the process according to the invention. This is intended purely as a possible procedure described by way of example without restricting the invention to its contents.

What is claimed is:

1. Double chamber container for holding and combining two separate components, at least one of which is liquid, comprising a cylindrical body (15) with a closure (20, 60) at each of the two ends (5, 70) of the body (15), an upper closure (60) at the liquid component end and a lower closure (20) at the solid component end, and a separating stopper (40) in the cylindrical body (15) as a seal between the two chambers (30, 50), the separating stopper (40) having a height H and the upper closure (60) at the liquid component end being movable by the application of pressure, and comprising on its underside a hollow needle (45) with a length L having at least one opening (42) which is provided at the end opposite the tip, where H<L.

2. Double chamber container according to claim 1, characterized in that the chamber (50) contains a liquid component and the other chamber (30) contains a solid component.

3. Double chamber container according to claim 1, characterized in that the solid component is a medicament, particularly a lyophilized medicament.

4. Double chamber container according to claim 1, characterized in that the liquid component is a dissolving or dispersing medium for the solid component.

5. Double chamber container according to claim 1, characterized in that the hollow needle (45) has an inner diameter of about 0.2 mm to about 1.6 mm.

6. Double chamber container according to claim 1, characterized in that the hollow needle (45) has a plurality of openings (42).

7. Double chamber container according to claim 1, characterized in that the opening(s) (42) and the internal diameter of the hollow needle (45) are selected such that the speed of passage of liquid component into the chamber (30) is adjustable.

8. Double chamber container according to claim 1, characterized in that the opening(s) (42) have a diameter in the range from about 0.2 mm to about 1.4 mm.

9. Double chamber container according to claim 1, characterized in that at the closure (60) and/or at the separating stopper (40), spacers and/or stops are provided on the hollow needle (45), which impede or prevent total penetration of the opening(s) (42) of the hollow needle (45) into the separating stopper (40).

10. Process for filling the double chamber container according to claim 1, comprising the steps of:
(1) sealing a cylindrical body (15) at the lower end (5);
(2) filling the cylindrical body (15) with a lyophilization solution;
(3) lyophilizing the cylindrical body in the lyophilizer to obtain a lyophilizate cake (30);
(4) putting a separating stopper (40) above the lyophilizate cake (30), the separating stopper (40) having a height H;
(5) filling the cylindrical body (15) with reconstitution medium (50) above the separating stopper;
(6) sealing the cylindrical body (15) with a closure comprising a hollow needle (45), wherein the closure (60) is movable at the liquid component end by the application of pressure and comprises, on its underside, a hollow needle (45) with a length L having at least one opening (42) which is provided at the end opposite the tip, where H<L, and the hollow needle (45) is directed towards the separating stopper (40).

11. Process according to claim 10, characterized in that the hollow needle (45) is mounted in the centre of the closure (60).

12. Process according to claim 10, characterized in that a hollow needle (45) is used having an internal diameter of about 0.2 mm to about 1.6 mm.

13. Process according to claim 10, characterized in that a hollow needle (45) with a plurality of openings 42 is used.

14. Process according to claim 10, characterized in that the opening(s) (42) and the internal diameter of the hollow needle (45) are selected so that the speed of passage of the liquid component into the chamber (30) is adjustable.

15. Process according to claim 10, characterized in that spacers are provided on the closure (60) or on the separating stopper (40) or stops are provided on the hollow needle (45) that prevent the opening or openings (42) of the hollow needle (45) from penetrating fully into the separating stopper (40).

16. Process for mixing two separate components, at least one of which is liquid, in a double chamber container according to one of the preceding claims 1 to 19, comprising
a cylindrical body (15) having a closure (20, 60) at each of the two ends (5, 70) of the body (15), an upper closure (60) on the liquid component side and a lower closure (20) on the solid component side, and
a separating stopper (40) in the cylindrical body (15) as a seal between the two chambers (30, 50),
the separating stopper (40) having a height H and the upper closure (60) being movable at the liquid component side by the application of pressure and comprising on its underside a hollow needle (45) of length L with at least one opening (42) which is mounted at the end opposite the tip, where H is <L,
comprising the steps of:
exerting pressure on the displaceable upper closure (60) until the hollow needle (45) has fully penetrated the separating stopper (40) and
allowing the liquid component to overflow through the opening(s) (42) and the hollow needle (45) to the solid component thereby obtaining a mixture.

17. Process according to claim 16, characterized in that the opening(s) (42) are at least partly and preferably totally left during the mixing of the two components in the chamber (50).

18. Process according to claim 16, characterized in that pressure is exerted until the closure (60) rests on the separating stopper (40).

19. Process according to claim 16, characterized in that during the mixing process the cylindrical body (15) is held vertically with the closure (20) at the bottom.

20. Process according to claim 16, characterized in that the pressure is exerted on the displaceable upper closure (60) using a punch.

21. Process according to claim 16, characterized in that the separating stopper (50) is forced through the hollow needle (45) onto the solid component during the penetration.

22. Process according to claim 16, characterized in that the hollow needle (45) for mixing is pressed into the separating stopper (40) until the opening(s) (42) is still at least partly contained within the chamber (50) containing the liquid component.

23. Process according to claim 16, characterized in that spacers are provided on the closure (60) or separating stopper (40) or stops are provided on the hollow needle (45) to impede or prevent total penetration of the opening(s) (42) into the separating stopper (40).
